(19) United States
(12) Patent Application Publication
(10) Pub. No.: US 2022/0310242 A1
(43) Pub. Date: Sep. 29, 2022

(54) SYSTEM AND METHOD FOR FLEET MANAGEMENT OF PORTABLE OXYGEN CONCENTRATORS

(71) Applicant: ResMed Pty Ltd, Bella Vista NSW (AU)

(72) Inventor: Hadley White, San Diego, CA (US)

(21) Appl. No.: 17/597,146

(22) PCT Filed: Jun. 26, 2020
(86) PCT No.: PCT/IB2020/056086

§ 371 Date: Dec. 27, 2021

Related U.S. Application Data
(60) Provisional application No. 62/867,650, filed on Jun. 27, 2019.

(51) Int. Cl. G16H 40/40; G06Q 10/04; B01D 53/047; B01D 53/30

(52) U.S. Cl. CPC G16H 40/40; G06Q 10/04; B01D 53/047; B01D 53/30; B01D 2253/108; B01D 2256/12; B01D 2257/102; B01D 2259/4541; B01D 2259/4533; A61M 16/101

(57) ABSTRACT

A system and method for prediction of the time to service components for a fleet of portable oxygen concentrators (POCs) is disclosed. Each of the POCs include a transmitter to transmit operational data. A network interface is configured to receive operational data from the POCs. A user database contains profiles of users associated with respective POCs. An analysis engine updates the profile of a user associated with a POC in the user database based on received operational data from the POC. The analysis engine determines a similar profile in the user database to the updated profile. The analysis engine predicts a service date for the component of the POC based on the similar profile and the updated profile.

### PRIORITY CLAIM

This application claims the benefit of and priority to U.S. Provisional Application No. 62/867,650, filed Jun. 27, 2019, and PCT Application No. PCT/IB2020/056086, filed Jun. 26, 2020, which are both hereby incorporated by reference herein in their entirety

### TECHNICAL FIELD

The present disclosure relates generally to portable oxygen concentrators (POCs), and more specifically for a system that predicts and supplies service dates for components for a fleet of POCs.

### BACKGROUND

There are many users that require supplemental oxygen as part of Long Term Oxygen Therapy (LTOT). Currently, the vast majority of users that are receiving LTOT are diagnosed under the general category of Chronic Obstructive Pulmonary Disease (COPD). This general diagnosis includes such common diseases as chronic bronchitis, emphysema, and related pulmonary conditions. Other users may also require supplemental oxygen, for example, obese individuals to maintain elevated activity levels, users with cystic fibrosis or infants with broncho-pulmonary dysplasia.

Doctors may prescribe oxygen concentrators or portable tanks of medical oxygen for these users. Usually a specific continuous oxygen flow rate is prescribed (e.g., 1 litre per minute (LPM), 2 LPM, 3 LPM, etc.). Experts in this field have also recognized that exercise for these users provides long term benefits that slow the progression of the disease, improve quality of life and extend user longevity. Most stationary forms of exercise like tread mills and stationary bicycles, however, are too strenuous for these users. As a result, the need for mobility has long been recognized. Until recently, this mobility has been facilitated by the use of small compressed oxygen tanks. The disadvantage of these tanks is that they have a finite amount of oxygen and they are heavy, weighing about 50 pounds, when mounted on a cart with dolly wheels.

Oxygen concentrators have been in use for about 50 years to supply users suffering from respiratory insufficiency with supplemental oxygen via oxygen enriched gas. Traditional oxygen concentrators used to provide these flow rates have been bulky and heavy, making ordinary ambulatory activities with them difficult and impractical. Recently, companies that manufacture large stationary home oxygen concentrators began developing portable oxygen concentrators (POCs). The advantage of POCs is that they can produce a theoretically endless supply of oxygen enriched gas. In order to make these devices small for mobility, the various systems necessary for the production of oxygen enriched gas are condensed.

Oxygen concentrators may take advantage of pressure swing adsorption (PSA). Pressure swing adsorption involves using a compressor to increase gas pressure inside a canister known as a sieve bed, that contains particles of a gas separation adsorbent that attracts nitrogen more strongly than it does oxygen. Ambient air usually includes approximately 78% nitrogen and 21% oxygen with the balance comprised of argon, carbon dioxide, water vapor and other trace gases. If a feed gas mixture such as air, for example, is passed under pressure through a sieve bed, part or all of the nitrogen will be adsorbed by the sieve bed, and the gas coming out of the vessel will be enriched in oxygen. When the sieve bed reaches the end of its capacity to adsorb nitrogen, it can be regenerated by reducing the pressure, thereby releasing the adsorbed nitrogen. It is then ready for another “PSA cycle” of producing oxygen enriched gas. By alternating canisters in a two-canister system, one canister can be concentrating oxygen (the so-called “adsorption phase”) while the other canister is being purged (the “purge phase”). This alternation results in a continuous separation of the oxygen from the nitrogen. In this manner, oxygen can be continuously concentrated out of the air for a variety of uses include providing supplemental oxygen to users. Further details regarding oxygen concentrators may be found, for example, in U.S. Published Patent Application No. 2009-0065007, published Mar. 12, 2009, and entitled “Oxygen Concentrator Apparatus and Method”, which is incorporated herein by reference.

The gas separation adsorbents used in POCs have a very high affinity for water. This affinity is so high that it overcomes nitrogen affinity, and thus when both water vapor and nitrogen are available in a feed gas stream (such as ambient air), the adsorbent will preferentially adsorb water vapor over nitrogen. Furthermore, when it is adsorbed, water does not desorb as easily as nitrogen. As a result, water molecules remain adsorbed even after regeneration and thus block the adsorption sites for nitrogen. Therefore, over time and use, water accumulates on the adsorbent, which becomes less and less efficient for nitrogen adsorption, to the point where the sieve bed needs to be replaced because no further oxygen concentration can take place. Such sieve beds may be referred to as exhausted or deactivated.

Other components also may require replacement such as the components of the compressor, inlet mufflers, batteries, and filters. Certain entities such as health care providers or POC manufacturers are responsible for large fleets of POCs and their respective users. The replacement of components such as the filter, the sieve bed, and the compressor for each of the POCs in the fleet is a consideration that must be addressed by the provider. In order to maximize efficiency and maintain operation, it is desirable to predict servicing of POCs as far in advance as possible. Currently service businesses learn of a POC failure when an alarm goes off on the device and they receive a call from the user. The alarm typically indicates either that an immediate service is needed or that it one will be needed within days. It is difficult to anticipate such service calls, which prevents orderly planning and scheduling to maximize service resources.

A need therefore exists for a POC manufacturer or service provider to be able to schedule the servicing of components of a fleet of POCs more efficiently.

### SUMMARY

Disclosed is a predictive system for servicing of components in a POC fleet. The system collects data from a fleet of POCs to increasingly precisely predict service dates for components on similar groups of POCs and their users.

One disclosed example is a system for predicting a service date for a component of a first portable oxygen concentrator (POC). The first POC includes a transmitter configured to transmit operational data of the first POC. The system includes a network interface configured to receive operational data from a plurality of POCs including the first POC. A user database contains profiles of users associated with respective POCs of the plurality of POCs. An analysis engine is operative to update a profile of a user associated with the first POC in the user database based on received operational data from the first POC. The analysis engine is operative to extract from the user database a profile of a second POC that is similar to the first POC, and predict a service date for the component of the first POC based on the profile of the second POC and the updated profile of the first POC.

A further implementation of the example system is an embodiment where each profile of a POC of the plurality of POCs comprises usage data for the POC. Another implementation is where the received operational data comprise usage data for the first POC. Another implementation is where the updating includes adding the usage data to the profile. Another implementation is where each profile of a POC includes geographic information for the POC. Another implementation is where the received operational data include location data associated with the usage data for the first POC. Another implementation is where the updating includes retrieving geographic information based on the location data, and adding the retrieved geographic information to the profile. Another implementation is where the geographic information includes at least one of humidity, air quality, and altitude. Another implementation is where each profile of a POC includes manufacturer data for the POC. Another implementation is where the analysis engine receives manufacturer data associated with a POC, and creates a profile for the associated POC comprising the manufacturer data. Another implementation is where the updating includes augmenting a deterioration curve based on the usage data. Another implementation is where the predicting includes estimating, based on the deterioration curves of the profiles, the service date. Another implementation is where the component is a sieve bed module of the POC, and the deterioration curve relates a remaining capacity of a sieve bed in the sieve bed module to the usage data. Another implementation is where the component is a component of a compression system of the POC, and the deterioration curve relates to a characteristic pressure of the compression system to the usage data. Another implementation is where the predicting includes estimating, based on the deterioration curves, a confidence interval around the estimated service date. Another implementation is where the analysis engine compares a size of the estimated confidence interval with a predetermined threshold. Another implementation is where the analysis engine creates, based on the comparing, a service schedule for the plurality of POCs from the predicted service date.

Another disclosed example is a method for predicting a service date for a component of a first portable oxygen concentrator (POC). The first POC includes a transmitter. Operational data are received from a plurality of POCs including the first POC through a network interface. The profile of a user associated with the first POC is updated in a user database based on the received operational data from the first POC. At least one similar profile of a second POC that is similar to the first POC is extracted from the user database. A service date for the component of the first POC is predicted based on the profile of the second POC and the updated profile of the first POC.

A further implementation of the example method is an embodiment where each profile of a POC includes usage data for the POC. Another implementation is where the received operational data include usage data for the first POC. Another implementation is where the updating includes adding the usage data to the profile. Another implementation is where each profile of a POC includes geographic information for the POC. Another implementation is where the received operational data include location data associated with the usage data for the first POC. Another implementation is where the updating includes retrieving geographic information based on the location data, and adding the retrieved geographic information to the profile. Another implementation is where the geographic information includes at least one of humidity, air quality, and altitude. Another implementation is where each profile of a POC includes manufacturer data for the POC. Another implementation is where the method further includes receiving manufacturer data associated with a POC, and creating a profile for the associated POC comprising the manufacturer data. Another implementation is where the updating includes augmenting a deterioration curve based on the usage data. Another implementation is where the predicting includes estimating, based on the deterioration curves of the profiles, the service date. Another implementation is where the component is a sieve bed module of the POC, and the deterioration curve relates a remaining capacity of a sieve bed in the sieve bed module to the usage data. Another implementation is where the component is a component of a compression system of the POC, and the deterioration curve relates a characteristic pressure of the compression system to the usage data. Another implementation is where the predicting includes estimating, based on the deterioration curves, a confidence interval around the estimated service date. Another implementation is where the method includes comparing a size of the estimated confidence interval with a predetermined threshold. Another implementation is where the method includes creating, based on the comparing, a service schedule for the plurality of POCs from the predicted service date.

Another disclosed example is a computer program product comprising instructions which, when executed by a computer, cause the computer to carry out the above described methods. Another implementation of the example computer program product is where the computer program product is a non-transitory computer readable medium.

Another disclosed example is a system that predicts the time required for replacing components for a plurality of portable oxygen concentrators (POCs). Each of the POCs includes a transmitter to transmit operational data on oxygen produced by the POCs. The system includes a network interface to collect operational data from each of the POCs. A user database stores user data for users associated with each of the POCs of the plurality of POCs. An analysis engine is operative to determine similar users according to the user data and the operational data collected from each of the POCs. The analysis engine determines service related data according to the user data and operational data. The analysis engine creates a POC profile for one subset of POCs of the plurality of POCs based on the service related data. The analysis engine predicts a service date to replace a component of the POCs in the subset of the POCs based on the POC profile.

A further implementation of the example system is an embodiment where the analysis engine receives operational data from a new POC, matches the new POC to the subset of POCs based on the received operational data, and provides the service date to replace a component for the new POC. Another implementation is where the component is one of a group comprising a compressor part, a sieve bed module for separating oxygen for the user of the POC, a battery, and a filter. Another implementation is where the prediction is based on times and date of use of the subset of POCs. Another implementation is where the prediction is based on the environment surrounding the subset of POCs. Another implementation is where the environment includes at least one of altitude, humidity and air quality. Another implementation is where the prediction is based on a manufacturing batch of the subset of POCs. Another implementation is where the analysis engine creates the profile for POCs from the manufacturing batch of the subset of POCs. Another implementation is where the analysis engine updates a delivery date of a replacement component in accordance with the prediction. Another implementation is where the system includes an ordering engine that communicates scheduling information to a supply system to supply replacement components for each of the subsets of the plurality of POCs. The analysis engine provides the prediction to the ordering engine. Another implementation is where each POC transmits an identification number unique to the POC to the analysis engine. Another implementation is where the analysis engine is operable for tracking short-term service of each of the POCs through a remaining capacity degradation curve based on the operational data. Another implementation is where the oxygen output of each POC is derived from operational data from the POCs and the profile of the subset of the POCs. Another implementation is where the operational data include one of pump pressure or oxygen flow output.

Another disclosed example is a method that predicts the time required for replacing components for a plurality of portable oxygen concentrators (POCs). Each of the POCs includes a transmitter to transmit operational data on oxygen produced by the POCs. Operational data from each of the POCs are collected via a network interface. User data for users associated with each of the POCs of the plurality of POCs is stored in a user database. Similar users according to the user data and the operational data collected from each of the POCs are identified. Service related data are determined according to the user data and the operational data. A POC profile for one subset of POCs of the plurality of POCs is created based on the service related data. A service date to replace a component of the POCs in the subset of the POCs is predicted based on the POC profile.

A further implementation of the example method is an embodiment where the method includes receiving operational data from a new POC, matching the new POC to the subset of POCs based on the received operational data, and providing the service date to replace a component for the new POC. Another implementation is where the component is one of the group comprising a compressor part, a sieve bed module for separating oxygen for the user of the POC, a battery, or a filter. Another implementation is where the prediction is based on times and date of use of the subset of POCs. Another implementation is where the prediction is based on the environment surrounding the subset of POCs. Another implementation is where the environment includes at least one of altitude, humidity and air quality. Another implementation is where the prediction is based on a manufacturing batch of the subset of POCs. Another implementation is where the profile is created from the manufacturing batch of the subset of POCs. Another implementation is where the method includes updating a delivery date of a replacement component in accordance with the prediction. Another implementation is where the method includes communicating the prediction to a supply system, and communicating scheduling information to the supply system to supply replacement components for each of the subsets of the plurality of POCs. Another implementation is where each POC transmits an identification number unique to the POC. Another implementation is where the method includes tracking short-term service of each of the POCs through a remaining capacity degradation curve based on the operational data. Another implementation is where the oxygen output of each POC is derived from operational data from the POCs and the profile of the subset of the POCs. Another implementation is where the operational data include one of pump pressure or oxygen flow output.

Another disclosed example is a computer program product comprising instructions which, when executed by a computer, cause the computer to carry out the above described methods. Another implementation is where the computer program product is a non-transitory computer readable medium.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 depicts a schematic diagram of the components of an oxygen concentrator;

FIG. 2 depicts a side view of examples of main components of an oxygen concentrator;

FIG. 3 depicts a schematic diagram of the outlet components of an oxygen concentrator;

FIG. 4 depicts a system of an example fleet data collection and management system that may be implemented for a fleet of oxygen concentrators including the oxygen concentrator in FIG. 1;

FIGS. 5A and 5B make up a flow diagram of a routine to collect data from a POC fleet and predict fleet component service dates; and

FIG. 6 shows an example deterioration curve of remaining capacity versus usage time for a sieve bed.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a system that allows entities servicing fleets of POCs to automatically optimize the scheduling of servicing and delivery of replacement components for cost and efficiency. This is especially valuable for those entities servicing POCs across a large geographic area and/or with a large number of POCs in their fleet. It also minimizes the chance of a user being deprived of a POC during an unexpected interruption due to predictable component failure.

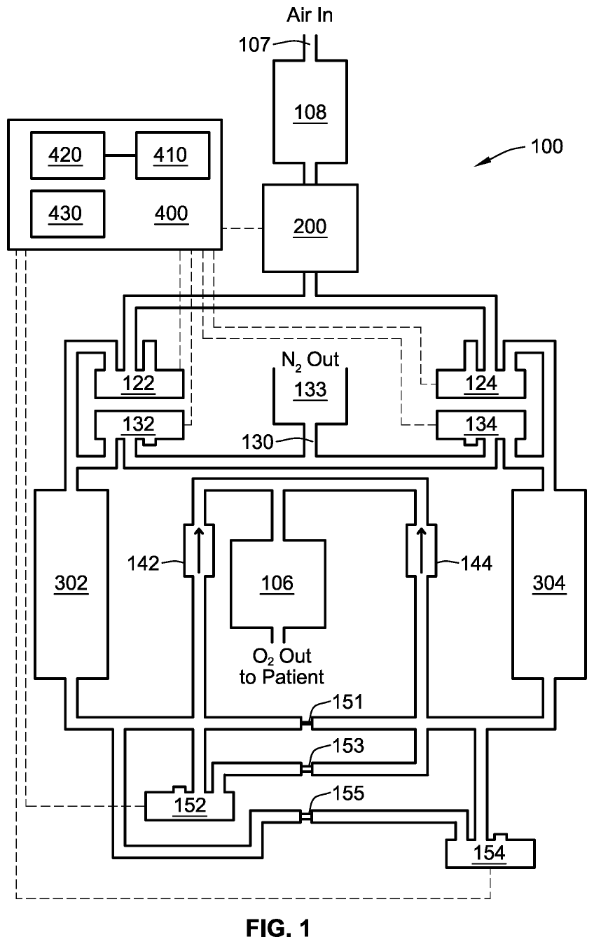

FIG. 1 illustrates a schematic diagram of an oxygen concentrator 100, according to an embodiment. Oxygen concentrator 100 may concentrate oxygen out of an air stream to provide oxygen enriched gas to a user. As used herein, “oxygen enriched gas” is composed of at least about 50% oxygen, at least about 60% oxygen, at least about 70% oxygen, at least about 80% oxygen, at least about 90% oxygen, at least about 95% oxygen, at least about 98% oxygen, or at least about 99% oxygen.

Oxygen concentrator 100 may be a portable oxygen concentrator. For example, oxygen concentrator 100 may have a weight and size that allows the oxygen concentrator to be carried by hand and/or in a carrying case. In one embodiment, oxygen concentrator 100 has a weight of less than about 20 lbs., less than about 15 lbs., less than about 10 lbs, or less than about 5 lbs. In an embodiment, oxygen concentrator 100 has a volume of less than about 1000 cubic inches, less than about 750 cubic inches; less than about 500 cubic inches, less than about 250 cubic inches, or less than about 200 cubic inches.

Oxygen may be collected from a feed gas by pressurising the feed gas in canisters 302 and 304, which contain a gas separation adsorbent. Gas separation adsorbents useful in an oxygen concentrator are capable of separating at least nitrogen from an air stream to leave oxygen enriched gas. Examples of gas separation adsorbents include compounds that are capable of separation of nitrogen from an air stream. Examples of adsorbents that may be used in an oxygen concentrator include, but are not limited to, zeolites (natural) or synthetic crystalline aluminosilicates that separate nitrogen from oxygen in an air stream under elevated pressure. Examples of synthetic crystalline aluminosilicates that may be used include, but are not limited to: OXYSIV adsorbents available from UOP LLC, Des Plaines, IL; SYLOBEAD adsorbents available from W. R. Grace & Co, Columbia, Md.; SILIPORITE adsorbents available from CECA S.A. of Paris, France; ZEOCHEM adsorbents available from Zeochem AG, Uetikon, Switzerland; and AgLiLSX adsorbent available from Air Products and Chemicals, Inc., Allentown, Pa.

As shown in FIG. 1, air may enter the oxygen concentrator through air inlet 107. Air may be drawn into air inlet 107 by compression system 200. Compression system 200 may draw in air from the surroundings of the oxygen concentrator and compress the air, forcing the compressed air into one or both canisters 302 and 304. In an embodiment, an inlet muffler 108 may be coupled to air inlet 107 to reduce sound produced by air being pulled into the oxygen concentrator by compression system 200. In an embodiment, inlet muffler 108 may be a moisture and sound absorbing muffler. For example, a water absorbent material (such as a polymer water absorbent material or a zeolite material) may be used to both absorb water from the incoming air and to reduce the sound of the air passing into the air inlet 107.

Compression system 200 may include one or more compressors capable of compressing air. Pressurized air, produced by compression system 200, may be forced into one or both of the canisters 302 and 304. In some embodiments, the feed gas may be pressurized in the canisters to a pressure approximately in a range of up to 30 pounds per square inch (psi). Other pressures may also be used, depending on the type of gas separation adsorbent disposed in the canisters.

Coupled to each canister 302/304 are inlet valves 122/124 and outlet valves 132/134. As shown in FIG. 1, inlet valve 122 is coupled to canister 302 and inlet valve 124 is coupled to canister 304. Outlet valve 132 is coupled to canister 302 and outlet valve 134 is coupled to canister 304. Inlet valves 122/124 are used to control the passage of air from compression system 200 to the respective canisters. Outlet valves 132/134 are used to release gas from the respective canisters during a venting process. In some embodiments, inlet valves 122/124 and outlet valves 132/134 may be silicon plunger solenoid valves. Other types of valves, however, may be used. Plunger valves offer advantages over other kinds of valves by being quiet and having low leakage.

In some embodiments, a two-step valve actuation voltage may be used to control inlet valves 122/124 and outlet valves 132/134. For example, a high voltage (e.g., 24 V) may be applied to an inlet valve to open the inlet valve. The voltage may then be reduced (e.g., to 7 V) to keep the inlet valve open. Using less voltage to keep a valve open may use less power (Power=Voltage * Current). This reduction in voltage minimizes heat build-up and power consumption to extend run time from the battery. When the power is cut off to the valve, it closes by spring action. In some embodiments, the voltage may be applied as a function of time that is not necessarily a stepped response (e.g., a curved downward voltage between an initial 24 V and a final 7 V).

In an embodiment, pressurized air is fed into one of canisters 302 or 304 while the other canister is being depressurized. For example, during use, inlet valve 122 is opened while inlet valve 124 is closed. Pressurized air from compression system 200 is forced into canister 302, while being inhibited from entering canister 304 by inlet valve 124. In an embodiment, a controller 400 is electrically coupled to valves 122, 124, 132, and 134. Controller 400 includes one or more processors 410 operable to execute program instructions stored in memory 420. The program instructions are operable to perform various predefined methods that are used to operate the oxygen concentrator. Controller 400 may include program instructions for operating inlet valves 122 and 124 out of phase with each other, i.e., when one of inlet valves 122 or 124 is opened, the other valve is closed. During pressurization of canister 302, outlet valve 132 is closed and outlet valve 134 is opened. Similar to the inlet valves, outlet valves 132 and 134 are operated out of phase with each other. In some embodiments, the voltages and the duration of the voltages used to open the input and output valves may be controlled by controller 400. The controller 400 may include a transmitter/receiver (transceiver) module 430 that may communicate with external devices to report data collected by the processor 410 or receive instructions and/or data from an external device for the processor 410.

Check valves 142 and 144 are coupled to canisters 302 and 304, respectively. Check valves 142 and 144 are one-way valves that are passively operated by the pressure differentials that occur as the canisters are pressurized and vented. Check valves 142 and 144 are coupled to canisters to allow oxygen enriched gas produced during pressurization of the canister to flow out of the canister, and to inhibit back flow of oxygen enriched gas or any other gases into the canister. In this manner, check valves 142 and 144 act as one-way valves allowing oxygen enriched gas to exit the respective canister while pressurized.

The term “check valve”, as used herein, refers to a valve that allows flow of a fluid (gas or liquid) in one direction and inhibits back flow of the fluid. Examples of check valves that are suitable for use include, but are not limited to: a ball check valve; a diaphragm check valve; a butterfly check valve; a swing check valve; a duckbill valve; and a lift check valve. Under pressure, nitrogen molecules in the pressurized feed gas are adsorbed by the gas separation adsorbent in the pressurized canister. As the pressure increases, more nitrogen is adsorbed until the gas in the canister is enriched in oxygen. The non-adsorbed gas molecules (mainly oxygen) flow out of the pressurized canister when the pressure difference across the check valve coupled to the canister reaches a value sufficient to overcome the resistance of the check valve. In one embodiment, the pressure drop of the check valve in the forward direction is less than 1 psi. The break pressure in the reverse direction is greater than 100 psi. It should be understood, however, that modification of one or more components would alter the operating parameters of these valves. If the forward flow pressure is increased, there is, generally, a reduction in oxygen enriched gas production. If the break pressure for reverse flow is reduced or set too low, there is, generally, a reduction in oxygen enriched gas pressure.

In an exemplary embodiment, canister 302 is pressurized by compressed air produced in compression system 200 and passed into canister 302. During pressurization of canister 302, inlet valve 122 is open, outlet valve 132 is closed, inlet valve 124 is closed and outlet valve 134 is open. Outlet valve 134 is opened when outlet valve 132 is closed to allow substantially simultaneous venting of canister 304 while canister 302 is pressurized. Canister 302 is pressurized until the pressure in canister 302 is sufficient to open check valve 142. Oxygen enriched gas produced in canister 302 exits through check valve 142 and, in one embodiment, is collected in an accumulator.

After some time, the gas separation adsorbent will become saturated with nitrogen and will be unable to separate significant amounts of nitrogen from incoming air. In the embodiment described above, when the gas separation adsorbent in canister 302 reaches this saturation point, the inflow of compressed air is stopped and canister 302 is vented to remove nitrogen. During venting, inlet valve 122 is closed, and outlet valve 132 is opened. While canister 302 is being vented, canister 304 is pressurized to produce oxygen enriched gas in the same manner described above. Pressurization of canister 304 is achieved by closing outlet valve 134 and opening inlet valve 124. The oxygen enriched gas exits canister 304 through check valve 144.

During venting of canister 302, outlet valve 132 is opened allowing pressurized gas (mainly nitrogen) to exit the canister through concentrator outlet 130. In an embodiment, the vented gases may be directed through muffler 133 to reduce the noise produced by releasing the pressurized gas from the canister. As gas is released from canister 302, the pressure in the canister drops, allowing the nitrogen to become desorbed from the gas separation adsorbent. The released nitrogen exits the canister through outlet 130, resetting the canister to a state that allows renewed separation of oxygen from an air stream. Muffler 133 may include open cell foam (or another material) to muffle the sound of the gas leaving the oxygen concentrator. In some embodiments, the combined muffling components/techniques for the input of air and the output of gas, may provide for oxygen concentrator operation at a sound level below 50 decibels.

During venting of the canisters, it is advantageous that at least a majority of the nitrogen is removed. In an embodiment, at least about 50%, at least about 60%, at least about 70%, at least about 80%, at least about 90%, at least about 95%, at least about 98%, or substantially all of the nitrogen in a canister is removed before the canister is re-used to separate oxygen from air. In some embodiments, a canister may be further purged of nitrogen using an oxygen enriched stream that is introduced into the canister from the other canister.

In an exemplary embodiment, a portion of the oxygen enriched gas may be transferred from canister 302 to canister 304 when canister 304 is being vented of nitrogen. Transfer of oxygen enriched gas from canister 302 to 304 during venting of canister 304 helps to further purge nitrogen (and other gases) from the canister. In an embodiment, oxygen enriched gas may travel through flow restrictors 151, 153, and 155 between the two canisters. Flow restrictor 151 may be a trickle flow restrictor. Flow restrictor 151, for example, may be a 0.009D flow restrictor (e.g., the flow restrictor has a radius 0.009″ which is less than the diameter of the tube it is inside). Flow restrictors 153 and 155 may be 0.013D flow restrictors. Other flow restrictor types and sizes are also contemplated and may be used depending on the specific configuration and tubing used to couple the canisters. In some embodiments, the flow restrictors may be press fit flow restrictors that restrict air flow by introducing a narrower diameter in their respective tube. In some embodiments, the press fit flow restrictors may be made of sapphire, metal or plastic (other materials are also contemplated).

Flow of oxygen enriched gas is also controlled by use of valve 152 and valve 154. Valves 152 and 154 may be opened for a short duration during the venting process (and may be closed otherwise) to prevent excessive oxygen loss out of the purging canister. Other durations are also contemplated. In an exemplary embodiment, canister 302 is being vented and it is desirable to purge canister 302 by passing a portion of the oxygen enriched gas being produced in canister 304 into canister 302. A portion of oxygen enriched gas, upon pressurization of canister 304, will pass through flow restrictor 151 into canister 302 during venting of canister 302. Additional oxygen enriched gas is passed into canister 302, from canister 304, through valve 154 and flow restrictor 155. Valve 152 may remain closed during the transfer process, or may be opened if additional oxygen enriched gas is needed. The selection of appropriate flow restrictors 151 and 155, coupled with controlled opening of valve 154 allows a controlled amount of oxygen enriched gas to be sent from canister 304 to 302. In an embodiment, the controlled amount of oxygen enriched gas is an amount sufficient to purge canister 302 and minimize the loss of oxygen enriched gas through venting valve 132 of canister 302. While this embodiment describes venting of canister 302, it should be understood that the same process can be used to vent canister 304 using flow restrictor 151, valve 152 and flow restrictor 153.

The pair of equalization/vent valves 152/154 work with flow restrictors 153 and 155 to optimize the air flow balance between the two canisters. This may allow for better flow control for venting the canisters with oxygen enriched gas from the other of the canisters. It may also provide better flow direction between the two canisters. It has been found that, while flow valves 152/154 may be operated as bi-directional valves, the flow rate through such valves varies depending on the direction of fluid flowing through the valve. For example, oxygen enriched gas flowing from canister 304 toward canister 302 has a flow rate faster through valve 152 than the flow rate of oxygen enriched gas flowing from canister 302 toward canister 304 through valve 152. If a single valve was to be used, eventually either too much or too little oxygen enriched gas would be sent between the canisters and the canisters would, over time, begin to produce different amounts of oxygen enriched gas. Use of opposing valves and flow restrictors on parallel air pathways may equalize the flow pattern of the oxygen between the two canisters. Equalising the flow may allow for a steady amount of oxygen to be available to the user over multiple cycles and also may allow a predictable volume of oxygen to purge the other of the canisters. In some embodiments, the air pathway may not have restrictors but may instead have a valve with a built-in resistance or the air pathway itself may have a narrow radius to provide resistance.

At times, oxygen concentrator may be shut down for a period of time. When an oxygen concentrator is shut down, the temperature inside the canisters may drop as a result of the loss of adiabatic heat from the compression system. As the temperature drops, the volume occupied by the gases inside the canisters will drop. Cooling of the canisters may lead to a negative pressure in the canisters. Valves (e.g., valves 122, 124, 132, and 134) leading to and from the canisters are dynamically sealed rather than hermetically sealed. Thus, outside air may enter the canisters after shutdown to accommodate the pressure differential. When outside air enters the canisters, moisture from the outside air may condense inside the canister as the air cools. Condensation of water inside the canisters may lead to gradual degradation of the gas separation adsorbents, steadily reducing ability of the gas separation adsorbents to produce oxygen enriched gas.

In an embodiment, outside air may be inhibited from entering canisters after the oxygen concentrator is shut down by pressurising both canisters prior to shutdown. By storing the canisters under a positive pressure, the valves may be forced into a hermetically closed position by the internal pressure of the air in the canisters. In an embodiment, the pressure in the canisters, at shutdown, should be at least greater than ambient pressure. As used herein the term “ambient pressure” refers to the pressure of the surroundings in which the oxygen concentrator is located (e.g. the pressure inside a room, outside, in a plane, etc.). In an embodiment, the pressure in the canisters, at shutdown, is at least greater than standard atmospheric pressure (i.e., greater than 760 mmHg (Ton), 1 atm, 101,325 Pa). In an embodiment, the pressure in the canisters, at shutdown, is at least about 1.1 times greater than ambient pressure; is at least about 1.5 times greater than ambient pressure; or is at least about 2 times greater than ambient pressure.

In an embodiment, pressurization of the canisters may be achieved by directing pressurized air into each canister from the compression system and closing all valves to trap the pressurized air in the canisters. In an exemplary embodiment, when a shutdown sequence is initiated, inlet valves 122 and 124 are opened and outlet valves 132 and 134 are closed. Because inlet valves 122 and 124 are joined together by a common conduit, both canisters 302 and 304 may become pressurized as air and/or oxygen enriched gas from one canister may be transferred to the other canister. This situation may occur when the pathway between the compression system and the two inlet valves allows such transfer. Because the oxygen concentrator operates in an alternating pressurize/venting mode, at least one of the canisters should be in a pressurized state at any given time. In an alternate embodiment, the pressure may be increased in each canister by operation of compression system 200. When inlet valves 122 and 124 are opened, pressure between canisters 302 and 304 will equalized however, the equalized pressure in either canister may not be sufficient to inhibit air from entering the canisters during shutdown. In order to ensure that air is inhibited from entering the canisters, compression system 200 may be operated for a time sufficient to increase the pressure inside both canisters to a level at least greater than ambient pressure. Regardless of the method of pressurization of the canisters, once the canisters are pressurized, inlet valves 122 and 124 are closed, trapping the pressurized air inside the canisters, which inhibits air from entering the canisters during the shutdown period.

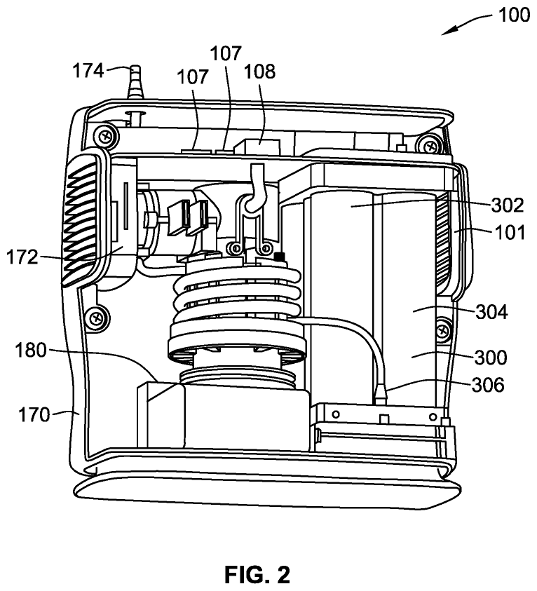

Referring to FIG. 2, an embodiment of an oxygen concentrator 100 is depicted. Oxygen concentrator 100 includes the compression system 200, a replaceable canister assembly 300, also referred to as a sieve bed module, having the canisters 302 and 304 in FIG. 1, and a power supply 180 (e.g. a battery) disposed within an outer housing 170. Inlets 101 are located in outer housing 170 to allow air from the environment to enter oxygen concentrator 100. Inlets 101 may allow air to flow into the compartment to assist with cooling of the components in the compartment. Power supply 180 provides a source of power for the oxygen concentrator 100. Compression system 200 draws air in through the inlet 107 and muffler 108. Muffler 108 may reduce noise of air being drawn in by the compression system and also may include a desiccant material to remove water from the incoming air. Oxygen concentrator 100 may further include fan 172 used to vent air and other gases from the oxygen concentrator. Outlet port 174 is used to attach a conduit to provide oxygen enriched air produced by the oxygen concentrator 100 to a user.

In some embodiments, compression system 200 includes one or more compressors. In another embodiment, compression system 200 includes a single compressor, coupled to all of the canisters of the canister system 300 via the inlet 306. The compression system 200 includes a compressor and a motor. The motor is coupled to the compressor and provides an operating force to the compressor to operate the compression mechanism. For example, the motor may be a motor providing a rotating component that causes cyclical motion of a component of the compressor that compresses air. When the compressor is a piston type compressor, the motor provides an operating force which causes the piston of the compressor to be reciprocated. Reciprocation of the piston causes compressed air to be produced by compressor. The pressure and flow rate of the compressed air are, in part, related to the speed that the compressor is operated at (e.g., how fast the piston is reciprocated). The motor may be a variable speed motor that is operable at various speeds to dynamically control the flow rate of air produced by compressor.

In one embodiment, the compressor may include a single head wobble type compressor having a piston. Other types of compressors may be used such as diaphragm compressors and other types of piston compressors. The motor may be a DC or AC motor and provides the operating power to the compressing component of the compressor. The motor may be a variable speed motor capable of operating the compressing component of compressor at variable speeds. The motor may be coupled to the controller 400 in FIG. 1, which sends operating signals to the motor to control the operation of the motor. For example, controller 400 may send signals to the motor to: turn the motor on, turn motor the off, and set the operating speed of the motor.

As the compressor components, such as the motor, seals, or pistons, wear during use, the ability of the compressor to compress air deteriorates. One measure of deterioration, which manifests, for example, in wear on the seals of the piston head, is a decrease in the pressure of the compressed air at a given motor speed, referred to as the characteristic pressure of the compressor. The POC 100 may include a sensor configured to monitor the characteristic pressure of the compression system 200 and provide a signal representative of the characteristic pressure to the controller 400. The pressure data may be taken periodically and stored to monitor the decrease in the characteristic pressure over time, thus indicating wearing of compressor components.

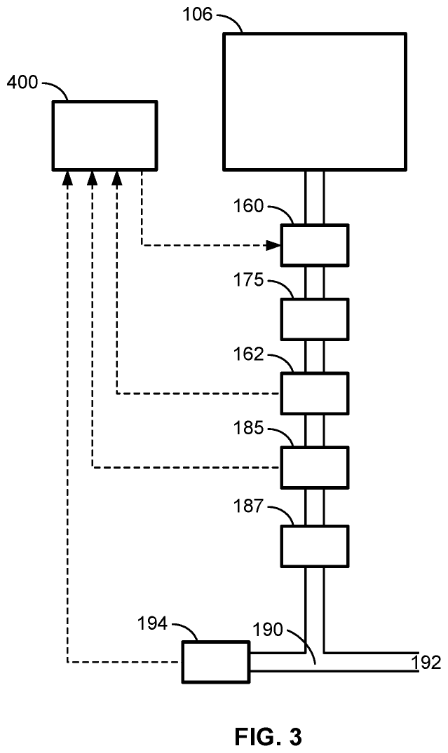

FIG. 3 shows the outlet of the oxygen concentrator 100 in FIG. 1. Oxygen enriched gas in an accumulator passes through a supply valve 160 via a flow restrictor 175 into an oxygen sensor 162 as depicted in FIG. 3. In an embodiment, the oxygen sensor 162 may include one or more devices for determining an oxygen concentration of gas passing through the chamber. Oxygen enriched gas then passes through a mass flow sensor 185 and a particulate filter 187.

The mass flow sensor 185 may be any sensor, or sensors, capable of estimating the mass flow rate of gas flowing through the conduit. Particulate filter 187 may filter bacteria, dust, granule particles, etc. prior to delivery of the oxygen enriched gas to the user. The oxygen enriched gas passes through the filter 187 to a connector 190 which sends the oxygen enriched gas to the user via a conduit 192 and to a pressure sensor 194. The oxygen enriched gas is delivered to the user via an airway delivery device, such as a nasal cannula, attached to the conduit 192.

The oxygen sensor 162 may be used to determine an oxygen concentration of gas passing through the sensor. The oxygen sensor 162 may be a chemical oxygen sensor, an ultrasonic oxygen sensor, or some other type of oxygen sensor.

The mass flow sensor 185 may be used to determine the mass flow rate of gas flowing through the outlet system. The mass flow sensor 185 may be coupled to controller 400. The mass flow rate of gas flowing through the outlet system may be an indication of the breathing volume of the user. Changes in the mass flow rate of gas flowing through the outlet system may also be used to determine a breathing rate of the user. The controller 400 may control actuation of supply valve 160 based on the breathing rate and/or breathing volume of the user, as estimated by mass flow sensor 185.

The airway delivery device is a component that also deteriorates over time and will ultimately need to be replaced. Deterioration of the airway delivery device may be indicated by increasing impedance, defined as the ratio of output pressure (as sensed by the output pressure sensor 194) to output flow rate (as sensed by the mass flow sensor 185).

Operation of oxygen concentrator 100 may be performed automatically using an internal controller such as the controller 400 coupled to various components of the oxygen concentrator 100, as described herein. Controller 400 includes one or more processors 410 and internal memory 420, as depicted in FIG. 1. Methods used to operate and monitor oxygen concentrator 100 may be implemented by program instructions stored in memory 420 or a carrier medium coupled to controller 400, and executed by one or more processors 410. A memory medium may include any of various types of memory devices or storage devices. The term “memory medium” is intended to include an installation medium, e.g., a Compact Disc Read Only Memory (CD-ROM), floppy disks, or tape device; a computer system memory or random access memory such as Dynamic Random Access Memory (DRAM), Double Data Rate Random Access Memory (DDR RAM), Static Random Access Memory (SRAM), Extended Data Out Random Access Memory (EDO RAM), Rambus Random Access Memory (RAM), etc.; or a non-volatile memory such as a magnetic media, e.g., a hard drive, flash memory, or optical storage. The memory medium may comprise other types of memory as well, or combinations thereof

In some embodiments, controller 400 includes processor 410 that includes, for example, one or more field programmable gate arrays (FPGAs), microcontrollers, etc. included on a circuit board disposed in oxygen concentrator 100. Processor 410 is capable of executing programming instructions stored in memory 420. In some embodiments, programming instructions may be built into processor 410 such that a memory external to the processor may not be separately accessed (i.e., the memory 420 may be internal to the processor 410).

Processor 410 may be coupled to various components of oxygen concentrator 100, including, but not limited to the compression system 200, one or more of the valves used to control fluid flow through the system (e.g., valves 122, 124, 132, 134, 152, 154, 160), oxygen sensor 162, pressure sensor 194, mass flow sensor 185, temperature sensor, cooling fans, humidity sensor, actigraphy sensor, altimeter, and any other component that may be electrically controlled or monitored. In some embodiments, a separate processor (and/or memory) may be coupled to one or more of the components.

The controller 400 is programmed to operate oxygen concentrator 100 and is further programmed to monitor the oxygen concentrator 100 for malfunction states. For example, in one embodiment, controller 400 is programmed to trigger an alarm if the system is operating and no breathing is detected by the user for a predetermined amount of time. For example, if controller 400 does not detect a breath for a period of 75 seconds, an alarm LED may be lit and/or an audible alarm may be sounded. If the user has truly stopped breathing, for example, during a sleep apnea episode, the alarm may be sufficient to awaken the user, causing the user to resume breathing. The action of breathing may be sufficient for controller 400 to reset this alarm function. Alternatively, if the system is accidently left on when output conduit 192 is removed from the user, the alarm may serve as a reminder for the user to turn oxygen concentrator 100 off to conserve power.

Controller 400 is further coupled to oxygen sensor 162, and may be programmed for continuous or periodic monitoring of the oxygen concentration of the oxygen enriched gas passing through oxygen sensor 162. A minimum oxygen concentration threshold may be programmed into controller 400, such that the controller lights an LED visual alarm and/or an audible alarm to warn the user of the low concentration of oxygen.

Controller 400 is also coupled to internal power supply 180 and is capable of monitoring the level of charge of the internal power supply. A minimum voltage and/or current threshold may be programmed into controller 400, such that the controller lights an LED visual alarm and/or an audible alarm to warn the user of low power condition. The alarms may be activated intermittently and at an increasing frequency as the battery approaches zero usable charge.

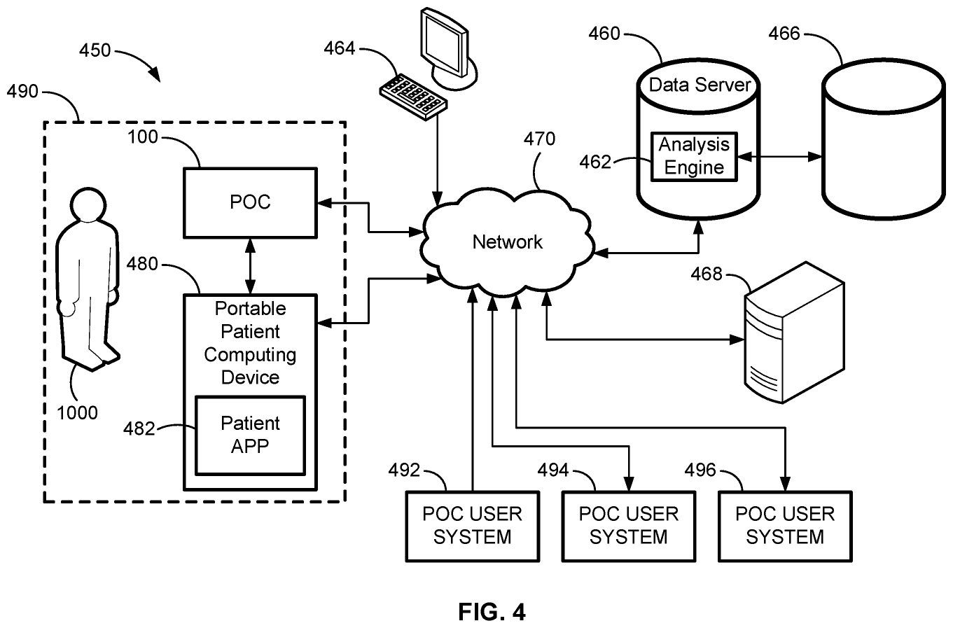

FIG. 4 illustrates one implementation of a connected oxygen therapy system 450, in which the controller 400 of the POC 100 includes the transceiver module 430 configured to allow the controller 400 to communicate, using a wireless communication protocol such as the Global System for Mobile Telephony (GSM) or other protocol (e.g., Wi-Fi), with a remote computing device such as a cloud-based server 460 over a network 470. The server 460 has a network interface enabling it to communicate over the network 470. The network 470 may be a wide-area network such as the Internet, or a local-area network such as an Ethernet. The controller 400 may also include a short range wireless module in the transceiver module 430 configured to enable the controller 400 to communicate, using a short range wireless communication protocol such as BluetoothTM, with a portable computing device 480 such as a smartphone. The smartphone 480 may be associated with a user 1000 of the POC 100.

The server 460 may also be in wireless communication with the portable computing device 480 using a wireless communication protocol such as GSM. A processor of the smartphone 480 may execute a program 482 known as an “app” to control the interaction of the smartphone with the POC 100 and/or the server 460.

The server 460 includes an analysis engine 462 that may execute operations such as a component service date prediction and a servicing routine, as will be explained below. The server 460 may also be in communication with other devices such as a personal computing device (workstation) 464 via a wired or wireless connection via the network 470. A processor of the personal computing device 464 may execute a “client” program to control the interaction of the personal computing device 464 with the server 460. One example of a client program is a browser. The server 460 has access to a database 466 that stores operational data about the POCs and users managed by the system 450. The database 466 may be segmented into individual databases such as a user database having information about users of the POCs and operational data associated with the POC use by the respective users, a manufacturer database including manufacturer data about the manufacture, transportation and storage of the POCs, and a reference database including deterioration curves, common profiles, and default servicing times. The deterioration curves could include, but are not limited to, time series of: oxygen concentration output from the sieve beds, remaining capacity of the sieve beds, characteristic pressure delivered by the compressor, flow rate output of the POC, internal humidity of the POC, battery recharge rate, leak flow rate of valves, impedance of the airway delivery device, and so on. Default servicing times (expected overall lifetimes) may be categorized by component with additional information in relation to the expected amount of use of the components in the POC. The server 460 may also be in communication via the network 470 with servers operated by other entities such as a supplier server 468 that coordinates the ordering and supply of replacement components for POCs.

The user 1000 of the POC, the POC 100 and portable computing device 480 may be organized as a POC user system 490. The connected oxygen therapy system 450 may comprise a plurality or “fleet” of POC user systems 490, 492, 494 and 496 that each include a POC user, a POC such as the POC 100, and a portable computing device such as the portable computing device 480. Each of the other POC user systems 492, 494 and 496 is in communication with the server 460, either directly or via respective portable computing devices associated with respective users of the POCs. The personal computing device 464 may be associated with a home medical equipment supplier (HME) that is responsible for the therapy of a population of users of the fleet of POCs. Other entities that may be associated with the personal computing device 464 with some responsibility for fleet management may be a manufacturer of the POC 100, a service business, or a health care professional or team of professionals.

The analysis engine 462 may implement machine-learning structures such as a neural network, decision tree ensemble, support vector machine, Bayesian network, or gradient boosting machine. Such structures can be configured to implement either linear or non-linear predictive models for component service dates. For example, data processing such as predicting service dates may be carried out by any one or more of supervised machine learning, deep learning, a convolutional neural network, and a recurrent neural network. In addition to descriptive and predictive supervised machine learning with hand-crafted features, it is possible to implement deep learning on the analysis engine 462. This typically relies on a larger amount of scored (labeled) data (such as many hundreds of data points from different POC devices) for normal and abnormal conditions. This approach may implement many interconnected layers of neurons to form a neural network (“deeper” than a simple neural network), such that more and more complex features are “learned” by each layer. Machine learning can use many more variables than hand-crafted features or simple decision trees.

Convolutional neural networks (CNNs) are used widely in audio and image processing for inferring information (such as for face recognition), and can also be applied to audio spectrograms, or even population scale genomic data sets created from the collected data represented as images. When carrying out image or spectrogram processing, the system cognitively “learns” temporal and frequency properties from intensity, spectral, and statistical estimates of the digitized image or spectrogram data.

In contrast to CNNs, not all problems can be represented with fixed-length inputs and outputs. Thus, the analysis can benefit from a system to store and use context information such as recurrent neural networks (RNNs) that can take the previous output or hidden states as inputs. In other words, they may be multilayered neural networks that can store information in context nodes. RNNs allow for processing of variable length inputs and outputs by maintaining state information across time steps, and may include LSTMs (long short term memories) types of “neurons” to enable RNNs increased control over the flow and mixing of inputs, which can be unidirectional or bidirectional,. [)] to manage the vanishing gradient problem and/or by using gradient clipping.

The analysis engine 462 may be trained for supervised learning of known service dates from known data inputs for assistance in analyzing input data. The analysis engine 462 may also be trained for unsupervised learning to determine unknown correlations between input data and service dates, to increase the range of analysis of the analysis engine 462.

Predictions of remaining usage times or service dates of POC components such as sieve beds, compressors, and airway delivery devices may be utilised by the various entities in the connected oxygen therapy system 450. In one implementation, the app 482 running on the portable computing device 480 could cause predicted remaining usage times or service dates of various POC components to be displayed on a display of the portable computing device 480. This could occur on the instruction of the server 460 via a “push notification” to the app, or on the initiative of the app itself.

In a further implementation, the server 460 may be configured to host a portal system. The portal system may receive, from the portable computing device 480 or directly from the POC 100, data relating to the operation of the POC 100. For example, such operational data may include estimates of remaining capacity of one or more of the sieve beds in a POC 100. As described above, the personal computing device 464 may execute a client application such as a browser to allow a user of the personal computing device 464 (such as a representative of an HME) to access the operational data of the POC 100, and other POCs in a connected oxygen therapy system 450, via the portal system hosted by the server 460. In this fashion, such a portal system may be utilised by an HME to manage a population of users of the fleet of POCs, e.g. the POC 100, or POC user systems 492, 494, and 496 in the connected oxygen therapy system 450. The HME may allow the data server 460 to provide supply information, such as the type of component, address of the user, convenient time of service, the ability or willingness of the user to do the service him or her self, etc., on the fleet of POCs to service entities by communicating component supply data to the supply entity server 468.

The portal system may provide actionable insights into user or device condition for the fleet of POCs and their users based on the operational data received by the portal system. Such insights may be based on rules that are applied to the operational data. In one implementation, the predicted remaining usage times or service dates of components of a fleet of POCs may be displayed to a representative of an HME on a display of a personal computing device 464 in a “window” of a client program interacting with the portal system. Further, a rule may be applied to each remaining usage time or service date prediction based on the status of the corresponding component. One example of such a rule is “If the remaining usage time for a POC component is less than three weeks, highlight the POC in the display of remaining usage times”. Application of such a rule to the remaining usage times results in the highlighting on the display of POCs with sieve beds approaching exhaustion or compressors near wearing out. The highlighted POCs may then be noted by the HME for imminent servicing. Another example of such a rule is “If the predicted service date for a POC component is less than three weeks away, highlight the POC in the display of predicted service dates”. Application of such a rule to the predicted service dates results in the highlighting on the display of POCs with sieve beds approaching exhaustion or compressors near wearing out. This is one example of the kind of rule-based fleet management made possible by the routine described below of predicting component service dates operating within the connected oxygen therapy system 450.

Optionally, such as in a case where the POC 100 determines an estimate of the remaining capacity of a sieve bed, the POC 100 may communicate a message, which may be based on the estimate, such as by a comparison with a threshold (e.g., if the estimate is at or below a threshold), to an external computing device of the system 450 such as to provide a notification message of a need for a replacement sieve bed for the POC 100. Such a message may comprise a request for a new sieve bed such as for arranging a purchase or replacement order for a new sieve bed via an ordering or fulfillment system implemented with any of the devices of FIG. 4 such as the supply entity server 468. Such a message may also be generated by any of the devices of the system 450 that receives either the remaining capacity estimate or the measurements and parameters necessary for determining the estimate. In such a case, the message may be further transmitted to other systems, such as a purchasing, ordering or fulfillment system or server(s) that may be configured to communicate with a device of the system 450 for arranging and/or completing such orders. Still further, in some implementations, the POC 100 may make a change in a control parameter of the POC 100 based on the estimate or a comparison of the estimate of remaining capacity and one or more thresholds. For example, one or more parameters for control of the PSA cycle of the POC 100 may be adjusted based on the comparison. Such adjustments may include, for example, to parameters for the various valve timings of the valves that control flow through the canisters for feed and purge cycles and/or compressor speed, etc. Such adjustments may be implemented for increasing remaining sieve bed usage time if a partially exhausted sieve bed is detected (e.g., less than 70%, 50% etc.) or resuming normal operating parameters for a detection of a replaced sieve bed (e.g., greater than 50% or at or near 100%).

Although each individual POC may monitor the need to service its own components, the system 450 also allows predictions of service dates for servicing components of entire groups of POCs of the fleet of POCs monitored by the system 450. Such economies of scale provide better servicing for the POC fleet managed by the system 450. Many HMEs or service businesses manage fleets of POCs in geographically disparate locations. This could be POC users spread across a state or nationally, or users in isolated areas that are expensive to access. By anticipating when individual POCs within a fleet are going to need to be serviced, it is possible to ‘cluster’ servicing to minimize staff and/or transportation costs. For example, POC A's sieve beds may be going to fail in 5 days, POC B's in 4 weeks and POC C's compressor in 7 weeks. Rather than servicing each POC individually in the days before failure (and making three trips), a business owner may choose to service all three at the same time because they are geographically distant from the service center but clustered near each other, and the salaried costs of the technician outweigh the costs of the replacement parts. When this logic is applied to fleets of tens of thousands of POCs the efficiency gains are significant.

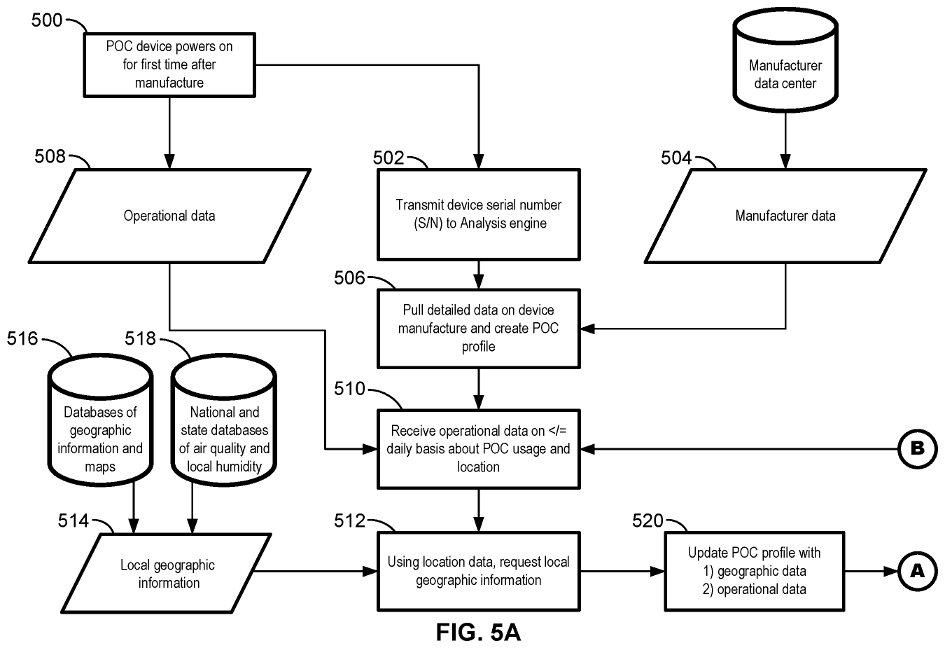

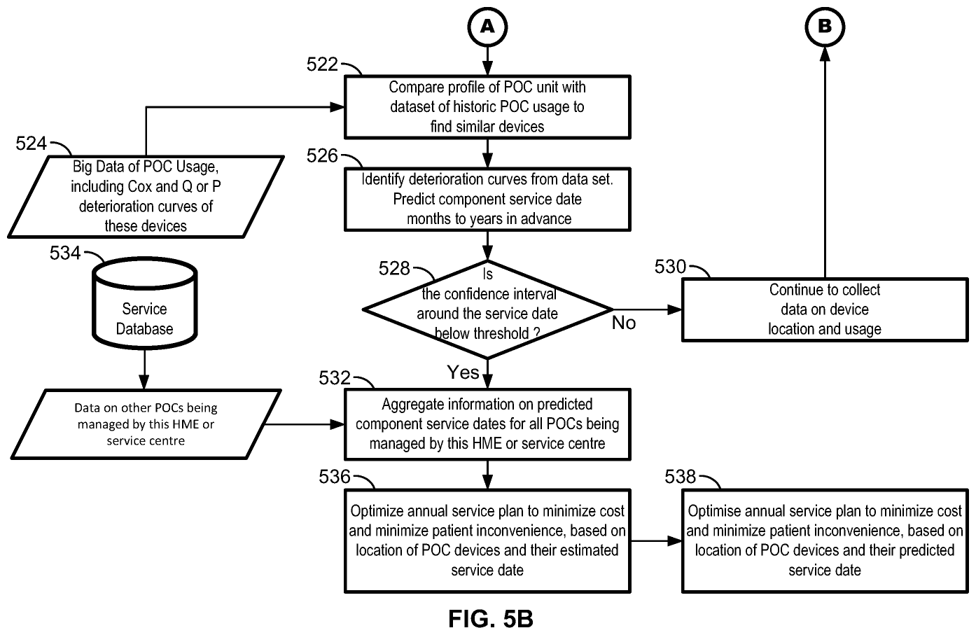

The flow diagram in FIGS. 5A and 5B is representative of an example routine implementable by machine readable instructions for the analysis engine 462 to predict component service dates for the POC user systems in the system 450 in FIG. 4. In this example, the machine readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine readable instructions represented by the flowcharts may be implemented manually. Further, although the example algorithm is described with reference to the flowchart illustrated in FIGS. 5A and 5B, persons of ordinary skill in the art will readily appreciate that many other methods of implementing the example machine readable instructions may alternatively be used. For example, the order of execution of the blocks may be changed, and/or some of the blocks described may be changed, eliminated, or combined.

The routine begins when the POC 100 is powered on for the first time after manufacture (500). The POC 100 transmits its unique device serial number (S/N) to the analysis engine 462 on the server 460 (502). As explained above, this may occur in direct communication with the POC 100 or through the portable computing device 480 in FIG. 4. The database 466 in FIG. 4 includes storage of data gathered from the manufacturer of the POC 100. Such manufacturer data may include the batch number, location of manufacture, time of manufacture of the POC 100, how it was transported from the manufacturing site to a local distribution center, and the time and location of storage at the distribution center. The database 466 in FIG. 4 also stores the received serial number of the POC 100. The database 466 associates the manufacturer data with the serial numbers of the POCs such as POC 100 in the fleet. The analysis engine 462 pulls the detailed manufacturer data (504) associated with the received serial number from the database 466 and creates a POC profile associated with the POC 100 (506) including the detailed manufacturer data. The POC profile contains the unique serial number and device information for the POC 100. The analysis engine 462 stores the new POC profile in the database 466 along with the POC profiles for the other POCs in the system 450.

On the first power up and subsequent power ups of the POC 100, operational data is gathered by the controller 400 on the POC 100 (508). Such operational data may include the output oxygen concentration, the remaining capacity of each sieve bed, the characteristic pressure of the compressor, the output flow rate, the time of day of use, the duration of use, and the geographic location of the POC 100 when used. An example method of estimating the remaining capacity of a sieve bed is disclosed in co-filed Patent Cooperation Treaty Application No. PCT/AU2020/050074, the entire contents of which are herein incorporated by reference. The location of the POC 100 may be obtained from geographical positioning data input to the POC 100 by the user, generated internally by a geolocation device within the POC 100, or taken directly from the portable computing device 480 in FIG. 4. The operational data are updated with each use of the POC 100. The operational data including usage data and location data are received by the analysis engine 462 periodically (510), e.g. on a daily basis or every 12 hours.

The routine takes the location data for the POC 100 received at step 510 and requests local geographic information for the location (512). The local geographic information (514), including altitude, local humidity, and local air quality, may be gathered from national and/or state and/or local databases of air quality and local humidity (516) and databases of geographic information such as altitudes (518). The routine then updates the POC profile with the operational data (usage data, remaining capacity data, etc.) and the geographic information (altitude, humidity, air quality) based on the location of the POC 100 (520) during usage. Updating the profile of a POC includes augmenting one or more deterioration curves for respective components of the POC. In one example of augmenting a deterioration curve, a further data point (current remaining capacity estimate and usage time) is added to a deterioration curve of remaining capacity versus usage time for each sieve bed of the POC.

The analysis engine 462 then compares the profile of the POC 100 with a dataset of historic POC usage comprising profile data from other POCs in the fleet (522). For example, POC # 1 was made with xyz zeolite batch, transported for 5 weeks on the sea and stored at a distribution center in Atlanta for 3 months. It is used in Tampa Fla., where the average annual humidity is 88.9%, usage is primarily at sea level, the pattern of usage is 2 hours a day during the week and 5 hours a day on the weekend, on setting 2 for 68% of the time and setting 3 for 32% of the time. The analysis engine 462 identifies similar POCs in its database 466, i.e. POCs that best match, or otherwise resemble, these manufacture and use conditions, and extracts the associated profile data of these similar POCs from the database 466 (522). For example, profile data may include deterioration curves of remaining sieve bed capacity, output flow rate (Q), or characteristic pressure (P) that may be stored in a database 524 that stores “big data” from numerous POC users. By analysing the profile(s) from this subset of data for a given component of the POC 100, the analysis engine 462 predicts the service date of the component (526). For example, in the case of the sieve bed module, a deterioration curve of remaining capacity vs usage time may be extracted from each similar POC profile and used to predict the service date of the sieve bed module. The analysis engine 462 may employ a machine-learning approach as described above to predict the service date.

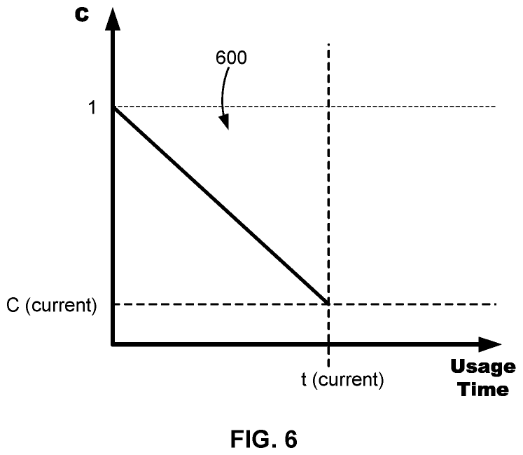

FIG. 6 shows an example deterioration curve 600 of remaining capacity C vs usage time for a sieve bed that may be used by the routine in FIGS. 5A and 5B. The deterioration curve 600 starts at a remaining capacity of 1 (100%) and decreases as usage time increases. While the curve in FIG. 6 is illustrated as linear, in general a deterioration curve will be of irregular profile. At the current usage time t (current), the remaining capacity is C (current).

Similarly, deterioration curves of characteristic pressure versus usage time may be extracted from the similar POC profiles and used to predict the date at which to service components of the compression system 200, such as the compressor motor, for example.

As the analysis engine 462 gathers more data on manufacture, location and duration of usage, the prediction of service date based on historic deterioration curves will become more precise. For example, after first ‘power up’ the analysis engine 462 may predict sieve bed servicing in 3-18 months. After the first week of usage and with some operational data, this may be a prediction of sieve bed servicing in 11-14 months, and after one month of usage and operational data this may be 12.3-12.7 months. This confidence interval, whose central value is the predicted date and whose size indicates the analysis engine's confidence in the predicted date, is calculated statistically based on the number of similar POCs in the database 466 and the elapsed time for collecting data.

The size of the confidence interval around the predicted service date is compared with a predetermined threshold value (528). When the confidence interval of the predicted service date falls below the threshold 1 month), the analysis engine 462 starts reporting the predicted service date, and feeding that information into a service optimization plan. Until this threshold is met the analysis engine 462 will continue to collect operational data (530) on the device location and usage to further refine the profile (returning to step 510).

The predicted service date allows a business servicing a fleet of POCs to plan its service schedule months or even up to a year in advance. For example, accurate service dates for sieve bed modules allow a service schedule for replacement of sieve beds modules of all POCs in the system 450 that fit a certain profile to be drawn up. Data collected from the fleet of POCs may enable an accurate prediction of the date to service components. Further, such predictive servicing may occur even when the POC fails to communicate additional operational data to the server 460.

If the size of the confidence interval of the predicted service date is less than the predetermined threshold (528), the analysis engine 462 aggregates information on predicted service dates for all POC user systems in the fleet being managed by the server 460 (532) from a service database (534) that includes the predicted sieve bed module and compressor service dates for all POCs serviced by an HME or service center. The analysis engine 462 then constructs an optimised service schedule to minimise cost to the HME and inconvenience to the users based on the location of the POCs in the fleet and their predicted service dates (536). Finally, the analysis engine 462 triggers execution of the optimised service schedule (538), which may include posting of replacement parts to users, recalling POCs or components for service, and dispatching technicians to POC locations. After each service of a component of a POC, the profile of the POC is updated in the database 466 with service data relating to the service, including the date of the service and the type of service.

The precision of the service date prediction routine executed by the analysis engine 462 becomes greater over time as more POC operational, manufacturer and service data are added to the profiles in the database 466. The reference database becomes bigger and therefore the predictive results become more refined. By comparison, current ad hoc service models are ‘dumb’ and do not get more precise with time.

The predictive data allow additional instructions to be provided to the controller 400 on the POC 100 to alter its operation so as to better fit within an optimized service schedule. For example, the controller 400 may increase compressor output to keep oxygen concentration consistent as the remaining capacity of one or more sieve beds decreases given normal usage of the POC based on the collected data. The controller 400 may also be instructed to regulate compressor output to conform to scheduling of service or delivery of replacement components.

Additional information in relation to a user's schedule may be used to allow predictive servicing of the POC without interrupting therapy. For example, even if a POC does not need to be serviced, the routine may provide service or supply replacement components at a more convenient time that will not interrupt therapy within a predetermined time of the scheduled needed service.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

### LABEL LIST

| | |
|---|---|
| oxygen concentrator | 100 |
| inlets | 101 |
| air inlet | 107 |
| muffler | 108 |
| inlet valve | 122 |
| valve | 124 |
| outlet | 130 |
| valve | 132 |
| muffler | 133 |
| valve | 134 |
| check valve | 142 |
| check valve | 144 |
| flow restrictor | 151 |
| valve | 152 |
| flow restrictor | 153 |
| valve | 154 |
| flow restrictor | 155 |
| valve | 160 |
| oxygen sensor | 162 |
| outer housing | 170 |
| fan | 172 |
| outlet port | 174 |
| flow restrictor | 175 |
| power supply | 180 |
| mass flow sensor | 185 |
| particulate filter | 187 |
| connector | 190 |
| conduit | 192 |
| pressure sensor | 194 |
| compression system | 200 |
| canister system | 300 |
| canister | 302 |
| canister | 304 |
| inlet | 306 |
| controller | 400 |
| processors | 410 |
| memory | 420 |
| transceiver module | 430 |
| connected oxygen therapy system | 450 |
| server | 460 |
| analysis engine | 462 |
| personal computing device | 464 |
| database | 466 |
| supplier server | 468 |
| network | 470 |
| portable computing device | 480 |
| app | 482 |
| POC user system | 490 |
| POC user system | 492 |
| POC user system | 494 |
| POC user system | 496 |
| step | 500 |
| data | 501 |
| detailed manufacturer data | 504 |
| step | 510 |
| step | 512 |
| local geographic information | 514 |
| database | 516 |
| step | 522 |
| database | 524 |
| step | 526 |
| step | 528 |
| step | 530 |
| service database | 534 |
| step | 536 |
| step | 538 |
| example deterioration curve | 600 |
| user | 1000 |

1. A system for predicting a service date for a component of a portable oxygen concentrator (POC), the system comprising:
a first POC including: a compression system; a sieve bed module including an adsorbent material accepting air from the compression system and producing oxygen enriched gas; a controller coupled to the compression system and sieve bed module to control the flow of oxygen enriched air; and a transmitter configured to transmit operational data relating to the operation of the compression system and the sieve bed module of the first POC, wherein the operational data include usage data of the POC, environmental data of the POC, and manufacturing batch data of the component of the POC;
a network interface configured to receive operational data from a plurality of POCs including the first POC;
a user database containing profiles of the plurality of POCs; and
a processor coupled to the network interface and user database, the processor operative to:
update a profile of the first POC in the user database based on received operational data from the first POC;
extract from the user database a profile of a second POC, the profile of the second POC including a service time for a corresponding component to the component of the first POC, the profile of the second POC having similar environmental data, manufacturer batch, and usage data to the first POC; and
predict a service date for the component of the first POC based on the profile of the second POC and the updated profile of the first POC.

2. The system of claim 1, wherein each profile of a POC of the plurality of POCs comprises usage data for the POC.

3-4. (canceled)

5. The system of claim 2, wherein:
each profile of a POC comprises geographic information for the POC, and
the received operational data comprises location data associated with the usage data for the first POC.

6. (canceled)

7. The system of claim 5, wherein the updating comprises:
retrieving geographic information based on the location data; and
adding the retrieved geographic information to the profile, wherein the geographic information is associated with the environmental data, wherein the environmental data includes at least one of humidity, air quality, and altitude.

8. (canceled)

9. The system of claim 2, wherein:
the updating comprises augmenting a deterioration curve based on the usage data, and
the predicting comprises estimating, based on the deterioration curves of the profiles, the service date.

10. (canceled)

11. The system of claim 9, wherein the component is the sieve bed module of the POC, and the deterioration curve relates a remaining capacity of a sieve bed in the sieve bed module to the usage data.

12. The system of claim 9, wherein the component is a component of the compression system of the POC, and the deterioration curve relates a characteristic pressure of the compression system to the usage data.

13. The system of claim 9, wherein the predicting comprises estimating, based on the deterioration curves, a confidence interval around the estimated service date.

14. The system of claim 13, wherein the processor is further operative to:
compare a size of the estimated confidence interval with a predetermined threshold, and
create, based on the comparing, a service schedule for the plurality of POCs from the predicted service date.

15-16. (canceled)

17. The system of claim 1, wherein the processor is further operative to:
receive manufacturer data associated with a POC; and
create a profile for the associated POC comprising the manufacturer data.

18. A method for predicting a service date for a component of a first portable oxygen concentrator (POC), the first POC including: a compression system; a sieve bed module including an adsorbent material accepting air from the compression system and producing oxygen enriched gas; a controller coupled to the compression system and sieve bed module to control the flow of oxygen enriched air; and a transmitter, the method comprising:
collecting operational data relating to the operation of the compression system and the sieve bed module of the first POC, wherein the operational data include usage data of the POC, environmental data of the POC, and manufacturing batch data of the component of the POC;
transmitting the operational data via the transmitter;
receiving operational data from a plurality of POCs including the first POC through a network interface;
updating a profile of the first POC in a user database based on the received operational data from the first POC;
extracting from the user database at least one profile of a second POC, the profile of the second POC including a service time for a corresponding component to the component of the first POC, the profile of the second POC having similar environmental data, manufacturer batch, and usage data to the first POC; and
predicting a service date for the component of the first POC based on the profile of the second POC and the updated profile of the first POC.

19. The method of claim 18, wherein each profile of a POC comprises usage data for the POC.

20-21. (canceled)

22. The method of claim 18, wherein:
each profile of a POC comprises geographic information for the POC, and
the received operational data comprise location data associated with the usage data for the first POC.

23. (canceled)

24. The method of claim 18, wherein the updating comprises:
retrieving geographic information based on the location data; and
adding the retrieved geographic information to the profile, wherein the geographic information is associated with the environmental data, wherein the environmental data includes at least one of humidity, air quality, and altitude.

25. (canceled)

26. The method of claim 18, wherein:
the updating comprises augmenting a deterioration curve based on the usage data, and
the predicting comprises estimating, based on the deterioration curves of the profiles, the service date.

27. (canceled)

28. The method of claim 26, wherein the component is the sieve bed module of the POC, and the deterioration curve relates a remaining capacity of a sieve bed in the sieve bed module to the usage data.

29. The method of claim 26, wherein the component is a component of the compression system of the POC, and the deterioration curve relates a characteristic pressure of the compression system to the usage data.

30. The method of claim 26, wherein the predicting comprises estimating, based on the deterioration curves, a confidence interval around the estimated service date, the method further comprising:
comparing a size of the estimated confidence interval with a predetermined threshold, and
creating, based on the comparing, a service schedule for the plurality of POCs from the predicted service date.

31-33. (canceled)

34. The method of claim 33, further comprising:
receiving manufacturer data associated with a POC; and
creating a profile for the associated POC comprising the manufacturer data.

35-66. (canceled)

67. A non-transitory computer readable medium comprising instructions which, when executed by a computer, cause the computer to carry out a method comprising:
receiving operational data from a plurality of portable oxygen concentrators (POCs) including a first POC through a network interface, wherein the first POC includes: a compression system; a sieve bed module including an adsorbent material accepting air from the compression system and producing oxygen enriched gas; a controller coupled to the compression system and sieve bed module to control the flow of oxygen enriched air; and a transmitter configured to transmit operational data relating to the operation of the compression system and the sieve bed module of the first POC, wherein the operational data include usage data of the POC, environmental data of the POC, and manufacturing batch data of the component of the POC;
updating a profile of the first POC in a user database based on the received operational data from the first POC;
extracting from the user database at least one profile of a second POC, the profile of the second POC including a service time for a corresponding component to the component of the first POC, the profile of the second POC having similar environmental data, manufacturer batch, and usage data to the first POC; and
predicting a service date for a component of the first POC based on the profile of the second POC and the updated profile of the first POC.
